(19) United States
(12) Patent Application Publication
(10) Pub. No.: US 2026/0185064 A1
(43) Pub. Date: Jul. 2, 2026

(54) APPLICATION OF HIGH-TEMPERATURE-TOLERANT TNPB NUCLEASE IN NUCLEIC ACID DETECTION AND GENE EDITING

(71) Applicants: Yazhouwan National Laboratory, Sanya (CN); Huazhong Agricultural University, Wuhan (CN)

(72) Inventors: Shuhong ZHAO, Sanya (CN); Changzhi ZHAO, Sanya (CN); Bingrong XU, Sanya (CN); Shengsong XIE, Wuhan (CN); Xinyun LI, Wuhan (CN); Sheng LI, Wuhan (CN); Xiaosong HAN, Sanya (CN)

(73) Assignees: Yazhouwan National Laboratory, Sanya (CN); Huazhong Agricultural University, Wuhan (CN)

(21) Appl. No.: 19/432,195

(22) Filed: Dec. 24, 2025

(30) Foreign Application Priority Data
Dec. 26, 2024 (CN) 202411932793.0

(51) Int. Cl. C12N 9/22; A61K 48/00; C12N 15/62; C12N 15/85

(52) U.S. Cl. CPC C12N 9/22; A61K 48/0041; C12N 15/62; C12N 15/85; C07K 2319/09; C12Y 301/21

(57) ABSTRACT

The TnpB nuclease is any one of (A1), (A2), and (A3): (A1) the TnpB nuclease having the amino acid sequence shown in SEQ ID NO: 1; (A2) a protein having more than 80% identity with the TnpB nuclease and having an identical biological function as the TnpB nuclease, where the protein is obtained by substituting, deleting, and/or adding one or more amino acid residues to the amino acid sequence of the TnpB nuclease; and (A3) a fusion protein obtained by fusing a protein tag to an N-terminus and/or a C-terminus of (A1) or (A2). The TnpB nuclease possesses both cis-cleavage activity and trans-cleavage activity and has broad application prospects in the fields of nucleic acid detection and gene editing.

### CROSS REFERENCE TO THE RELATED APPLICATIONS

This application is based upon and claims priority to Chinese Patent Application No. 202411932793.0, filed on Dec. 26, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBYJS073-PKG_SequenceListing.xml, created on Dec. 23, 2025, and is 52,487 bytes in size.

### TECHNICAL FIELD

The present invention relates to the technical field of gene editing, and in particular to an application of a programmable TnpB nuclease in gene editing.

### BACKGROUND

TnpB is an RNA-guided compact nuclease, approximately 400 amino acids in size, first discovered in bacterial transposons. The TnpB system mainly includes two key components: 1) the TnpB protein, responsible for recognizing and cleaving target DNA; and 2) the guide RNA (ωRNA). The ωRNA interacts with the TnpB protein, guiding it to recognize and cleave the target DNA sequence. Although numerous applications of the TnpB nuclease in gene editing have been reported both domestically and internationally, there are few reports in the field of nucleic acid detection. The TnpB protein is the ancestor of the clustered regularly interspaced short palindromic repeats-associated protein 12 (Cas12) protein in the clustered regularly interspaced short palindromic repeats (CRISPR) system of type V. The trans-cleavage activity of the Cas12 protein has been widely used in in vitro diagnostics, including for pathogenic microorganisms, toxins, and compounds. Therefore, the TnpB protein with trans-cleavage activity needs to be identified. The TnpB nuclease can have the gene editing activity improved through protein engineering and optimization of the ωRNA molecule. AlphaFold3 can accurately predict RNA-protein-DNA complexes. Further research is needed to determine whether engineering ωRNA molecule can improve TnpB nuclease activity.

### SUMMARY

The purpose of the present invention is to provide an application of a programmable TnpB nuclease in gene editing, thereby addressing the problems existing in the prior art. The TnpB nuclease provided by the present invention possesses both cis-cleavage activity and trans-cleavage activity, and has broad application prospects in the fields of nucleic acid detection and gene editing.

To achieve the above purpose, the present invention provides the following solution:

The present invention provides a TnpB nuclease, where the TnpB nuclease is any one of (A1), (A2), and (A3):
(A1) the TnpB nuclease having the amino acid sequence shown in SEQ ID NO: 1;
(A2) a protein having more than 80% identity with the TnpB nuclease and having an identical biological function as the TnpB nuclease, where the protein is obtained by substituting, deleting, and/or adding one or more amino acid residues to the amino acid sequence of the TnpB nuclease; and
(A3) a fusion protein obtained by fusing a protein tag to an N-terminus and/or a C-terminus of (A1) or (A2).

The present invention also provides a coding gene of the TnpB nuclease.

Furthermore, when the amino acid sequence of the TnpB nuclease is as shown in SEQ ID NO: 1, the nucleotide sequence of the coding gene is as shown in SEQ ID NO: 2.

The present invention also provides a biomaterial, where the biomaterial is any one of (B1)-(B3):
(B1) a gene expression cassette, including the coding gene;
(B2) a recombinant vector including the gene expression cassette; and
(B3) a recombinant host cell including the recombinant vector.

The present invention also provides an application of the coding gene or biomaterial in preparation of the TnpB nuclease.

The present invention also provides a composition for gene editing, including the TnpB nuclease and ωRNA; where:
the ωRNA includes a backbone portion and a targeting sequence portion; and
the targeting sequence portion is complementary to a target sequence of a target gene.

Furthermore, the nucleotide sequence of the backbone portion of the ωRNA is as shown in SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35.

The present invention also provides a gene editing system, including a recombinant vector expressing the TnpB nuclease and a recombinant vector expressing ωRNA; where:
the ωRNA includes a backbone portion and a targeting sequence portion;
the targeting sequence portion is complementary to a target sequence of a target gene; and
the nucleotide sequence of the backbone portion of the ωRNA is as shown in SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35.

The present invention also provides an application of the TnpB nuclease, composition, or gene editing system in nucleic acid recognition or gene editing for non-disease diagnosis and treatment purpose.

The gene editing includes gene modification, gene knockout, alteration of gene product expression, mutation repair, or insertion of polynucleotides in prokaryotic genome, eukaryotic genome, or in vitro gene.

The present invention also provides a method of gene editing, including a step of transfecting the gene editing system into a host cell to perform the gene editing.

The present invention discloses the following technical effects:
Through deep-mining of massive metagenomic sequencing data from public databases, the present invention discovered a novel TnpB nuclease (ISBce3_TnpB) from the ISBce3 transposon family. This protein is only 369 amino acids in size, recognizes the TAM sequence of TAC, and possesses both cis-cleavage activity and trans-cleavage activity, capable of trans-cleaving ssDNA and ssRNA.

The present invention further enhances gene editing activity by engineering the ωRNA sequence of the ISBce3_TnpB nuclease, showing broad application prospects in in vitro detection of pathogenic microorganisms, molecular diagnostics, and precise modification of mammalian genomes.

### BRIEF DESCRIPTION OF THE DRAWINGS

To more clearly illustrate the technical solutions in the embodiments of the present invention or the prior art, the drawings used in the embodiments will be briefly described below. Obviously, the drawings described below are merely some embodiments of the present invention. For those of ordinary skill in the art, other drawings can be obtained based on these drawings without any creative effort.

FIG. 1 is a schematic diagram of the domains of the ISBce3_TnpB nuclease.

FIG. 2 shows the results of agarose gel electrophoresis detection of the in vitro double-stranded DNA cleavage activity of the ISBce3_TnpB nuclease; where S represents substrate and P represents product.

FIG. 3 shows fluorescence intensity detection results of the ISBce3_TnpB nuclease trans-cleaving of the single-stranded DNA (ssDNA) reporter, when targeting double-stranded DNA (dsDNA) and ssDNA at different temperatures, respectively.

FIG. 4 shows fluorescence intensity detection results of the ISBce3_TnpB nuclease trans-cleaving of the ssDNA and ssRNA reporter, respectively, when targeting the dsDNA at different temperatures; where “+” indicates the addition of dsDNA, and “−” indicates the absence of dsDNA.

FIG. 5 shows fluorescence intensity detection results of the ISBce3_TnpB nuclease trans-cleaving of the ssDNA and ssRNA reporter, respectively, when targeting the ssDNA at different temperatures; where “+” indicates the addition of ssDNA, and “−” indicates the absence of ssDNA.

FIG. 6 shows fluorescence intensity detection results of the ISBce3_TnpB nuclease trans-cleaving of ssDNA-reporters of AAAAA, TTTTT, GGGGG, or CCCCC, respectively, when targeting the dsDNA at different temperatures; where “+” indicates the addition of dsDNA and “−” indicates the absence of dsDNA.

FIG. 7 shows detection results of the cis-cleavage activity of the ISBce3_TnpB nuclease after TAM mutation.

FIG. 8 shows detection results of the cis-cleavage activity of the ISBce3_TnpB nuclease after sequential mutation of the ωRNA sequence when targeting the same target.

FIG. 9 shows detection results of the trans-cleavage activity after 5′ truncation of the ωRNA when targeting the dsDNA and the ssDNA, respectively.

FIG. 10 shows detection results of the trans-cleavage activity after truncation of the Stem 1 sequence of 165-ωRNA.

FIG. 11 shows detection results of the trans-cleavage activity after truncation of the Stem 2 and Stem 3 sequences of 165-ωRNA.

FIG. 12 shows detection results of the trans-cleavage activity of two ωRNAs with lengths of 105 nt and 97 nt, which are obtained by combining the optimal ωRNAs truncated at the 5′ end, Stem1, Stem2, and Stem3.

FIGS. 13A-13B show results of evaluating the gene editing activity of ISBce3_TnpB nuclease in the HEK293T cells; where FIG. 13A represents detection results of the gene editing activity of wild-type ISBce3_TnpB nuclease in the HEK293T cells; FIG. 13B represents detection results of the gene editing activity of ISBce3_TnpB nuclease with engineered ωRNA in the HEK293T cells.

### DETAILED DESCRIPTION OF THE EMBODIMENTS

Various exemplary embodiments of the present invention are now described in detail. This detailed description should not be considered as a limitation of the present invention, but rather as a more detailed description of certain aspects, characteristics, and embodiments of the present invention.

It should be understood that the terminology used in the present invention is merely for describing specific embodiments and is not intended to limit the present invention. Furthermore, for numerical ranges in the present invention, it should be understood that each intermediate value between the upper limit and the lower limit of the range is also specifically disclosed. Any stated value or intermediate value within the stated range, as well as each smaller range between any other stated value or intermediate value within said range, is also included within the scope of the present invention. The upper limit and the lower limit of these smaller ranges may be independently included or excluded from the range.

Unless otherwise indicated, the nucleic acids are written from left to right in the 5′ to 3′ direction; the amino acid sequences are written from left to right in the direction from the N-terminus (amino terminus) to the C-terminus (carboxyl terminus).

### Terminology Explanation

Unless otherwise defined, all technical and scientific terms used herein have the same meaning as commonly understood by those of ordinary skill in the art to which the present invention pertains.

TnpB transposase: TnpB is a nuclease derived from bacteria and archaea, belonging to the CRISPR-associated protein family of types I-F. Initially discovered in transposons (mobile gene elements), it is thus named a transposase. TnpB plays a crucial role in gene editing, recognizing specific DNA sequences and performing cleavage or insertion at those locations.

ωRNA (RNA-guided nuclease activity factor): ωRNA is a small RNA molecule associated with TnpB, typically encoded by the host genome. It binds to the TnpB protein, forming a complex that recognizes and targets specific DNA sequences. This RNA-guided mechanism is similar to CRISPR RNA (crRNA) in the CRISPR-Cas system.

TAM (TnpB-Associated Motif): TAM is a specific DNA sequence recognized and bound by the TnpB nuclease. These sequences are typically located near the target gene and guide TnpB in precise cleavage or edit within the genome.

Cis-cleavage: This refers to the TnpB nuclease cleaving the target DNA sequence directly paired with it after binding to its ωRNA.

Trans-cleavage: This refers to the TnpB nuclease recognizing and binding to target DNA and cleaving not only the DNA sequence paired with the ωRNA but also adjacent or other DNA molecules.

While the present invention describes only preferred methods and materials, any methods and materials similar or equivalent to those described herein may be used in the implementation or testing of the present invention. All references to the present specification are incorporated by way of reference to disclose and describe the methods and/or materials associated with those references. In the event of any conflict with any incorporated reference, the contents of the present specification shall prevail.

Various modifications and variations can be made to the specific embodiments described in the specification of the present invention without departing from the scope or spirit of the present invention, which will be apparent to those skilled in the art. Other embodiments derived from the specification of the present invention will be apparent to those skilled in the art. The specification and embodiments of the present invention are exemplary only.

The terms “including”, “comprising”, “having”, “containing” etc., as used herein are open-ended terms, meaning they include but are not limited to.

### Example 1: Discovery of the Novel TnpB Nuclease Based on Metagenomics Strategy

Deep mining of bacterial-encoded proteins was performed using massive metagenomic sequencing data from public databases such as the NCBI nrpsd (Non-Redundant Protein Sequence Database) non-redundant protein database and the Global Microbial Gene Catalogue (GMGC) Database. The simplified workflow was as follows: For all contig sequences in the target database, minced and prodigal software were used to search for and locate REs (Right Element) and neighboring expressed proteins. Redundancy was then removed from these proteins using CD-hit software. Finally, mega software was used for protein clustering analysis, and hmmer software was used to identify and classify proteins with similar RuvC domains. Ultimately, a novel, unknown bacterial protein was identified. This protein is only 369 amino acids in size, and its amino acid sequence is shown in SEQ ID NO: 1. The nucleic acid sequence of its encoding gene is shown in SEQ ID NO: 2.

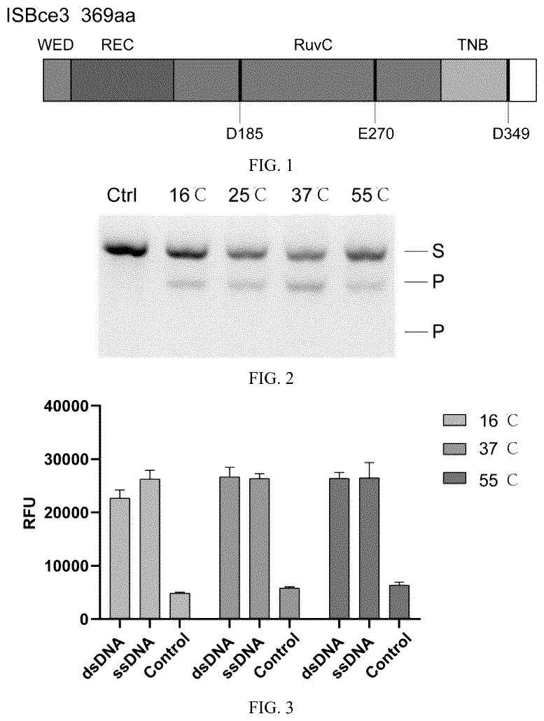

After determining the candidate protein, multiple sequence homology alignment was performed on the RNA sequence derived from the right-terminal transposon element of its genome to locate the transposon terminus. The corresponding ωRNA secondary structure was predicted using the RNAfold web server (rna.tbi.univie.ac.at/cgi-bin/RNAWebSuite/RNAfold.cgi) online website and compared with the secondary structures of known TnpB nuclease ωRNA to determine the sequence of candidate ωRNA. The structure of the ωRNA-protein-DNA complex was predicted using AlphaFold 3, and the predicted structural model was compared with the cryo-electron microscopy structure of the known ISDra2 nuclease to finally determine the domain composition and characteristics of the novel ISBce3_TnpB nuclease (as shown in FIG. 1).

### Example 2: ISBce3_TnpB Nuclease Exhibits Nucleic Acid Cleavage Activity In Vitro

In this example, the ISBce3_TnpB protein (the nucleic acid sequence of its E. coli codon-optimized coding gene is shown in SEQ ID NO: 3) was obtained through prokaryotic expression protein purification technology. Its corresponding RNA was obtained through in vitro transcription and purification, and the cleavage activity of the ISBce3_TnpB protein against dsDNA was tested in vitro at different temperatures. The ISBce3 protein was guided to recognize and bind to the target nucleic acid using ωRNA that pairs with the target nucleic acid, thereby activating its cleavage activity against the target nucleic acid. Then the agarose gel electrophoresis was performed to assess cleavage efficiency by observing changes in the size of the target band.

In this embodiment, the target dsDNA is a partial fragment of the ASFV p72 gene, with the nucleotide sequence shown in SEQ ID NO: 4. The nucleotide sequence of the corresponding ωRNA is shown in SEQ ID NO: 5.

SEQ ID NO: 4:
[]CTGTAACGCAGCACAGCTGAACCGTTCTGAAGAAGAAGAAAGTTAATA
GCAGATGCCGATACCACAAGATCAGCCGTAGTGATAGACCCCACGTAA
TCCGTGTCCCAACTAATATAAAATTCTCTTGCTCTGGATACGTTAATA
TGACCACTGGGTTGGTATTCCTCCCGTGGCTTCAAAGCAAAGGTAATC
ATCATCGCACCCGGATCATCGGGGGTTTTAATCGCATTGCCTCCGTAG
TGGAAGGGTATGTAAGAGCTGCAGAACTTTGATGGAAATTTATCGATA
AGATTGATACCATGAGCAGTTACGGAAATGTTTTTAATAATAGGTAAT
GTGATCGGATACGTAACGGGGCTAATATCAGATATAGATGAACATGCG
TCTGGAAGAGCTGTATCTCTATCCTGAAAGCTTATCTCTGCGTGGTGA
GTGGGCTGCATAATGGCGTTAACAACATGTCCGAACTTGTGCCAATCT
CGGTGTTGATGAGGATTTTGATCGGAGATGTTCCAGGTAGGTTTTAAT
CCTATAAACATATATTCAATGGGCCATTTAAGAGCAGACATTAGTTTT
TCATCGTGGTGGTTATTGTTGGTGTGGGTCACCTGCGTTTTATGGACA
CGTATCAGCGAAAAGCGAACGCGTTTTACAAAAAGGTTGTGTATTTCA
GGGGTTACAAACAGGTTATTGATGTAAAGTTCATTATTCGTGAGCGAG
ATTTCATTAATGACTCCTGGGATAAACCATGG;

the reverse complementary sequence of the region marked in bold is the TAM, and the reverse complementary sequence of the underlined sequence is the targeting sequence.

SEQ ID NO: 5:
GAAUAUUCGUUAUGCACCUGUGGUUGAUGGUAAAAGUCAAUCAGCAU
AGGGAACUAUAUGUUCUGCCCUAUGAGGGGUAAUGAGAUACCCUAAU
CUUGAGGACUGUUCAAAACAGAAAUGGACUGCGAACGCUUAGUCACU
CAAGAAUCCCACCCGUUAAACCGUAAGGUUUAGGGCUUGCGUCUUUA
GACGUGGGAGUCUCAACUUUGCUUUGAAGCCACGGG (The
underlined sequence is the targeting sequence).

First, using the previously constructed pMD18T-p72 plasmid (the p72 fragment was amplified from ASFV genomic DNA and ligated into the pMD-18T vector via T-A cloning to construct the pMD18T-p72 plasmid) as a template, and p72-F (SEQ ID NO: 6) and p72-R (SEQ ID NO: 7) as the primers, PCR amplification was performed to obtain the p72 gene fragment. Second, after codon optimization for E. coli, the DNA sequence encoding ISBce3_TnpB was synthesized, cloned into the pET-28a prokaryotic expression vector, transformed into E. coli strain BL21, and after identifying positive clones, isopropyl β-D-1-thiogalactopyranoside (IPTG)-induced expression was performed. The target protein was then purified by affinity chromatography. The in vitro cleavage reaction used the following system: 10×CutSmart Buffer of 2 μL, ISBce3_TnpB protein of 500 ng, ωRNA of 300 ng, and the purified product of p72 target amplification of 200 ng. The reaction mixture was incubated at 16° C., 25° C., 37° C., and 55° C. for 15 min, respectively. After the reaction was complete, proteinase K of 1 μL was added, and the reaction was terminated by incubation at 60° C. for 10 min. The control group did not receive any ωRNA. After the reaction, detection was performed by 1.5% agarose gel electrophoresis, and the cleavage activity of the novel ISBce3_TnpB nuclease at different temperatures was detected using the gel imaging system.

The results are shown in FIG. 2. Compared with the control group, the ISBce3_TnpB protein could cleave dsDNA at temperatures between 16° C. and 55° C. This indicates that the ISBce3_TnpB protein has targeted nucleic acid cleavage activity.

### Example 3: ISBce3_TnpB Activates Trans-Cleavage Activity by Targeting dsDNA or SSDNA

In this example, the targeting ability to dsDNA or ssDNA was evaluated using the complex-RNP formed by the ISBce3_TnpB protein and ωRNA. The dsDNA target and ωRNA were the same as in Example 2. The sequence of the ssDNA target is: 5′-CCCGTGGCTTCAAAGCAAAG-3′ (SEQ ID NO: 8).

The assay system of the trans-cleavage activity had a total volume of 20 μL, including 10×CutSmart Buffer of 2 μL, ISBce3_TnpB protein of 500 ng, ωRNA of 300 ng, 100 μM ssDNA-reporter (5′-ROX/GTATCCAGTGCG/BHQ2-3′) of 0.2 μL, where GTATCCAGTGCG is shown in SEQ ID NO: 47 and the purified fragment of the dsDNA target of 200 ng or ssDNA target of 5 μM. The control group contained neither dsDNA target nor ssDNA target. Three reaction temperatures were set: 16° C., 37° C., and 55° C. The reaction time was 60 min. After the reaction was complete, proteinase K of 1 μL was added to each reaction tube, and the reaction was terminated by incubation at 60° C. for 10 min. The resulting solution was transferred to a microplate plate, and diethyl pyrocarbonate (DEPC) water of 80 μL was added to each well for dilution. The fluorescence intensity of each reaction was read at 561-601 nm using a microplate reader.

The results are shown in FIG. 3. At the three set temperatures, the fluorescence signal values of ISBce3_TnpB nuclease were significantly higher than those of the control group when targeting dsDNA or ssDNA. This indicates that ISBce3_TnpB nuclease can activate trans-cleavage activity by targeting dsDNA or ssDNA.

### Example 4: Evaluation of the Reporter Type for Trans-Cleavage when ISBce3_TnpB Nuclease Targets dsDNA

In this example, the reporter type for trans-cleavage of the ISBce3_TnpB nuclease when targeting dsDNA was evaluated. ISBce3_TnpB protein was prepared using prokaryotic expression purification technology, ωRNA was prepared by in vitro transcription, and the dsDNA target was amplified by PCR and purified.

The dsDNA target and the ωRNA in this example were the same as in Example 2. The sequence of the ssDNA-reporter is 5′-ROX/GTATCCAGTGCG/BHQ2-3′, where GTATCCAGTGCG is shown in SEQ ID NO: 47 and the sequence of the ssRNA-reporter is 5′-ROX/GUAUCCAGUGCG/BHQ2-3′ where GUAUCCAGUGCG is shown in SEQ ID NO: 48.

The volume of the reaction system was 20 μL, including 10×CutSmart Buffer of 2 μL, ISBce3 protein of 500 ng, ωRNA of 300 ng, dsDNA target of 200 ng, and 100 μM ssDNA-reporter or ssRNA-reporter of 0.2 μL. No dsDNA target was added to the control group. Three temperatures were set: 16° C., 37° C., and 55° C. The reaction time was 60 min. After the reaction was complete, proteinase K of 1 μL was added to each reaction, and the reaction was terminated by incubation at 60° C. for 10 min. The resulting solution was transferred to a microplate plate, and DEPC water of 80 μL was added to each well for dilution. The fluorescence intensity of each reaction was read at 561-601 nm using a microplate reader.

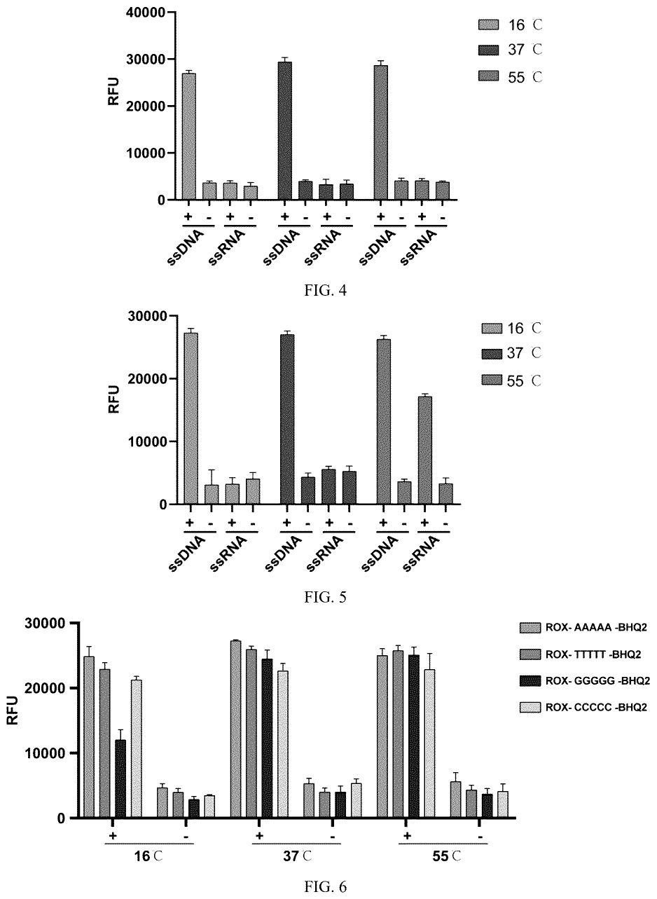

The results are shown in FIG. 4. At the three temperatures, only the ssDNA-reporter group produced a strong fluorescence signal. In summary, under conditions of 16° C.-55° C., the ISBce3_TnpB nuclease, when targeting dsDNA, can only trans-cleave the ssDNA-reporter.

### Example 5: Evaluation of the Reporter Type for the Trans-Cleavage when ISBce3_TnpB Nuclease Targets ssDNA

In this example, the reporter type for the trans-cleavage of the ISBce3_TnpB nuclease when targeting ssDNA was evaluated. ISBce3 protein was prepared using prokaryotic expression purification technology, and ωRNA was prepared by in vitro transcription.

The ssDNA target selected in this example was the same as in Example 3. The sequence of the ωRNA was the same as in Example 2. The sequences of ssDNA and RNA reporter were the same as in Example 4.

The volume of the reaction system was 20 μL, including 10×CutSmart Buffer of 2 μL, ISBce3_TnpB protein of 500 ng, ωRNA of 300 ng, synthesized ssDNA (10 μM) target of 0.5 μL, and 100 μM ssDNA reporter or ssRNA reporter of 0.2 μL. No ssDNA target was added to the control group. Three temperatures were set: 16° C., 37° C., and 55° C. The reaction time was 60 min. After the reaction was complete, proteinase K of 1 μL was added to each reaction, and the reaction was terminated by incubation at 60° C. for 10 min. The resulting solution was transferred to a microplate plate, and DEPC water of 80 μL was added to each well for dilution. The fluorescence intensity of each reaction was read at 561-601 nm using a microplate reader.

The results are shown in FIG. 5. When the ISBce3_TnpB nuclease targeted ssDNA, the ssDNA reporter group produced a strong fluorescence signal at all three temperatures. The RNA reporter group only produced a strong fluorescence signal at 55° C. In summary, ISBce3_TnpB, when targeting ssDNA, can trans-cleave the ssDNA reporter under conditions of 16° C.-55° C., and can also trans-cleave the RNA reporter at 55° C.

### Example 6: Evaluation of the Base Sequence Preference of ISBce3_TnpB Nuclease for Trans-Cleavage of ssDNA Reporter

The dsDNA target and ωRNA sequence used in this example were the same as in Example 2. The ssDNA reporter sequence was designed into four types: (1) 5′-ROX/AAAAA/BHQ2-3′; (2) 5′-ROX/TTTTT/BHQ2-3′; (3) 5′-ROX/GGGGG/BHQ2-3′; (4) 5′-ROX/CCCCC/BHQ2-3′.

The volume of the reaction system was 20 μL, including 10×CutSmart Buffer of 2 μL, ISBce3_TnpB protein of 500 ng, ωRNA of 300 ng, dsDNA target of 200 ng, and 100 μM ssDNA-reporter of 0.2 μL. No dsDNA target was added to the control group. Three temperatures were set: 16° C., 37° C., and 55° C. The reaction time was 60 min. After the reaction was complete, proteinase K of 1 μL was added to each reaction, and the reaction was terminated by incubation at 60° C. for 10 min. The resulting solution was transferred to a microplate plate, and DEPC water of 80 μL was added to each well for dilution. The fluorescence intensity of each reaction was read at 561-601 nm using a microplate reader.

The results are shown in FIG. 6, the ISBce3_TnpB nuclease can trans-cleave ssDNA reporters with sequences of AAAAA, TTTTT, CCCCC, or GGGGG in the temperature range of 16° C.-55° C., but it has lower trans-cleavage activity for ssDNA reporter with the sequence of GGGGG at 16° C.

### Example 7: Evaluation of ISBce3_TnpB Nuclease Compatibility with TAMs

This example evaluates the cis-cleavage activity of the ISBce3_TnpB nuclease in vitro using different types of TAMs. Specifically, the target nucleic acid selected in this example is the ASFV p72 gene. First, PCR amplification was performed using p72-F1/p72-R1 as the primer pair to obtain fragment 1. Then, fragment 2 was amplified using p72-F2/p72-R2 as the primer pair, and finally fragment 3 was amplified using p72-F3/p72-R3 as the primer pair. Using p72-F1 and p72-R3 as primers, and fragments 1, 2, and 3 as templates, PCR amplification was performed to obtain targets containing different TAMs. The sequence of the ωRNA used was the same as in Example 2.

The nucleotide sequences of p72-F1, p72-R1, p72-F2, p72-R2, p72-F3, and p72-R3 are shown in SEQ ID NOS: 9-14, respectively.

The total volume of the reaction system was 20 μL, including 10×CutSmart Buffer of 2 μL, ISBce3_TnpB protein of 500 ng, and ωRNA of 300 ng, and p72 amplification products with different types of TAMs of 200 ng were added, respectively. No ωRNA was added to the control group. The reaction temperature was set at 37° C., and the reaction time was 60 min. After the reaction was complete, proteinase K of 1 μL was added to each reaction, and the reaction was terminated by incubation at 60° C. for 10 min. The reaction products were detected by 1.5% agarose gel electrophoresis, and the cleavage efficiency was evaluated by calculating the gray scale value of the electrophoresis results using ImageJ software.

The results are shown in FIG. 7, changes to the 2nd, 3rd, and 4th bases of the TAM decreased cis-cleavage activity, while changes to the 1st base resulted in activity comparable to that of the wild type. This indicates that the last three bases of the ISBce3_TnpB nuclease TAM are crucial for cis-cleavage activity, while tolerance for the 1st base is relatively high.

### Example 8: Evaluation of Mismatch Tolerance Between Guide Sequence of the ISBce3_TnpB Nuclease and the dsDNA Target

In this example, bases 1-20 of the guide sequence of the ISBce3_TnpB nuclease were mutated sequentially, and the mismatch tolerance between the guide sequence and the dsDNA target was evaluated by detecting cis-cleavage activity. Specifically, the target nucleic acid selected in this example was the ASFV p72 gene. Fragments 1, 2, and 3 were obtained using the same primers as in Example 7. Using p72-F1 and p72-R3 as primers and fragments 1, 2, and 3 as templates, PCR amplification produced dsDNA targets with sequentially mismatched bases from nucleotides 1 to 20, respectively. The sequence of ωRNA used was the same as in Example 2. The volume of the reaction system was 20 μL, including 10×CutSmart Buffer of 2 μL, ISBce3_TnpB protein of 500 ng, ωRNA of 300 ng, and p72 amplification products with different TAMs of 200 ng were added, respectively. No ωRNA was added to the control group. The reaction temperature was set at 37° C., and the reaction time was 60 min. After the reaction was complete, proteinase K of 1 μL was added to each reaction tube, and the reaction was terminated by incubation at 60° C. for 10 min. The reaction products were detected by 1.5% agarose gel electrophoresis, and the cleavage efficiency was evaluated by calculating the grayscale value of the electrophoresis results using ImageJ software. The cleavage efficiency of the wild-type target was quantified as 1, and the proportion of the cleavage efficiency of sequentially mismatched targets at different positions relative to that of the wild-type target was calculated as the cleavage efficiency of sequentially mismatched targets at different positions.

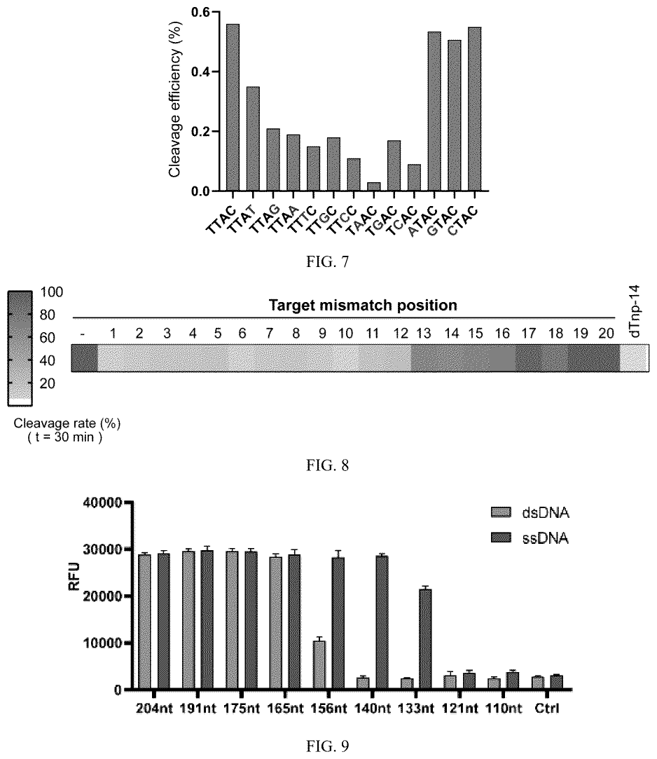

The results are shown in FIG. 8, compared with the wild-type and the catalytically inactivated ISBce3_TnpB variant (dTnpB), mismatched bases at positions 1-12 significantly reduced the cis-cleavage activity of ISBce3_TnpB. However, when the mismatched bases were at positions 13-20, high cis-cleavage activity was still observed. These results indicate that the cis-cleavage activity is activated when the first 1-12 bases of the guide sequence of the ISBce3_TnpB nuclease are perfectly paired with the target sequence.

### Example 9: Engineered ωRNA Scaffold Enhances the Trans-Cleavage Activity of ISBce3_TnpB Nuclease

In this example, the effect of truncated and optimized ωRNA scaffold sequences on the trans-cleavage activity of ISBce3_TnpB nuclease was evaluated. The wild-type ωRNA scaffold (WT-ωRNA) is 204 nt in length, and its nucleotide sequence is shown in SEQ ID NO: 15.

First, the sequence at 5′ end of the ωRNA scaffold was truncated to lengths of 191 nt (SEQ ID NO: 16), 175 nt (SEQ ID NO: 17), 165 nt (SEQ ID NO: 18), 156 nt (SEQ ID NO: 19), 140 nt (SEQ ID NO: 20), 133 nt (SEQ ID NO: 21), 121 nt (SEQ ID NO: 22), and 110 nt (SEQ ID NO: 23), respectively.

Specifically, in this embodiment, the selected dsDNA target nucleic acid is the CSFV E2 gene (the nucleotide sequence shown in SEQ ID NO: 24), with a TAM of 5′-TTAC-3′.

SEQ ID NO: 24:
ATCCACACCATCTGCATTGTCTTACTAGCCCCCCTGTGTAGACCACTGGT
TCACCTTTGACGCATGTCCAGTTGCCCCCCAACTTACAGTAGAATAAATC
CTCATTTTCCACCGTGGTGGTCACGCAATCCGTTCTATGCGAAGGCTTAT
CTCTCCTAAAGGTCTTTACCACTTCCGTTCTCAGAGTTGTAGGGCTCACC
GCTGTGCACTCTACGACACCCGTCCACCCTATTGGGCAGACAAGATAGAA
GGCACTGCCGTTCAATAAGGTTGTATTGTACTTCCCCTTGACAACAGGAC
TAGTATCGAAAGGGCACAGCCCGAACCCAAAGTCGTCTCCCATTTCCTCA
GTAGATGGGTTGGTCCCGTCGAACAAGAGCTCAAATGTTACAGACGTGGG
TAGAGCCCTCTTGTGTAATGATGCCAAGTACCTCCTGCTGACCACGCTGA
GTGCAGTGACTTTAAAGGAGCCCGCCACGCAAATAGCCTTGACGGTCCC
G;

the bolded marker sequence is TAM, and the underlined marker sequence is the target sequence. The selected ssDNA target sequence is a synthesized 20 nt oligonucleotide targeting for CSFV, with the sequence:

(SEQ ID NO: 25)
5′-CAACTCTGAGAACGGAAGTG-3′.

The volume of the reaction system was 20 μL, including 10×CutSmart Buffer of 2 μL, ISBce3_TnpB protein of 500 ng, different types of ωRNA variants of 300 ng, CSFV-E2 amplification product of 200 ng or synthesized 10 μM ssDNA target of 1 μL, and 100 μM ssDNA-reporter of 0.2 μL. No dsDNA target was added to the control group. The reactions were incubated at 37° C. for 5 min, 15 min, 30 min, 60 min, and 90 min, respectively. After the reaction was complete, proteinase K of 1 μL was added to each reaction, and the reaction was terminated by incubation at 60° C. for 10 min. The resulting solution was transferred to a microplate plate, and DEPC water of 80 μL was added to each well for dilution. The fluorescence intensity of each reaction was read at 561-601 nm using a microplate reader.

The results are shown in FIG. 9. When targeting dsDNA, when the length of the ωRNA was truncated to 156 nt, the activity of 156-ωRNA was significantly reduced compared to that of the WT-ωRNA, the activity of 140-ωRNA was lost, and the activities of 165-ωRNA, 175-ωRNA, and 191-ωRNA were similar to that of WT-ωRNA. When targeting ssDNA, the activity decreased when the length of the ωRNA was truncated to 133-ωRNA, and the activities of 140-ωRNA, 156-ωRNA, 165-ωRNA, 175-ωRNA, and 191-ωRNA were comparable to that of WT-ωRNA.

Therefore, the ISBce3_TnpB nuclease is more flexible in targeting ssDNA than in targeting dsDNA and is more tolerant of 5′ truncation of the ωRNA. In summary, based on the performance in targeting ssDNA and dsDNA, the 165-ωRNA is determined as the optimal ωRNA truncated at the 5′ end for subsequent experiments.

Following this, the three-dimensional structure of the TnpB-ωRNA-DNA complex was predicted using AlphaFold3, and the Stem 1 stem-loop region of the ωRNA scaffold was truncated to 150 nt (SEQ ID NO: 26), 139 nt (SEQ ID NO: 27), 138 nt (SEQ ID NO: 28), and 131 nt (SEQ ID NO: 29), respectively.

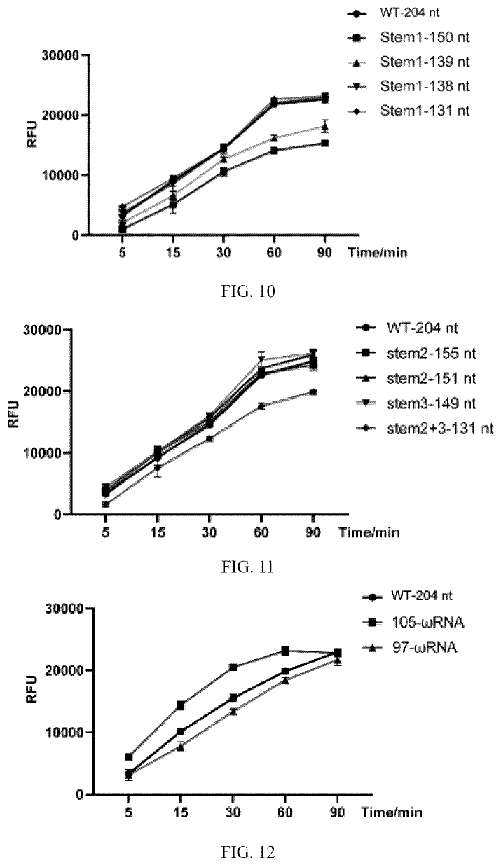

The results are shown in FIG. 10. The activities of Stem1-131-ωRNA and Stem1-138-ωRNA were similar to that of WT-ωRNA, while the activities of Stem1-150-ωRNA and Stem1-139-ωRNA were lower than that of WT-ωRNA. Therefore, Stem1-131-ωRNA is the optimal ωRNA truncated in the Stem1 region.

Then, the Stem2 and Stem3 regions were truncated using a similar method, resulting in Stem2-155-ωRNA (SEQ ID NO: 30), Stem2-151-ωRNA (SEQ ID NO: 31), Stem3-149-ωRNA (SEQ ID NO: 32), and Stem2 & Stem3-131-ωRNA (SEQ ID NO: 33), respectively.

The results are shown in FIG. 11. Compared to the wild type, Stem3-149-ωRNA exhibited the best activity, followed by Stem2-151-ωRNA and Stem2-155-ωRNA, while Stem2 & Stem3-131-ωRNA showed the lowest activity. Therefore, Stem2-155-ωRNA is the optimal ωRNA truncated in the Stem2 region, and Stem3-149-ωRNA is the optimal ωRNA truncated in the Stem3 region.

Furthermore, the optimal ωRNAs truncated at the 5′ end, Stem1, Stem2, and Stem3 were combined to obtain two ωRNAs with lengths of 105 nt (SEQ ID NO: 34) and 97 nt (SEQ ID NO: 35), respectively.

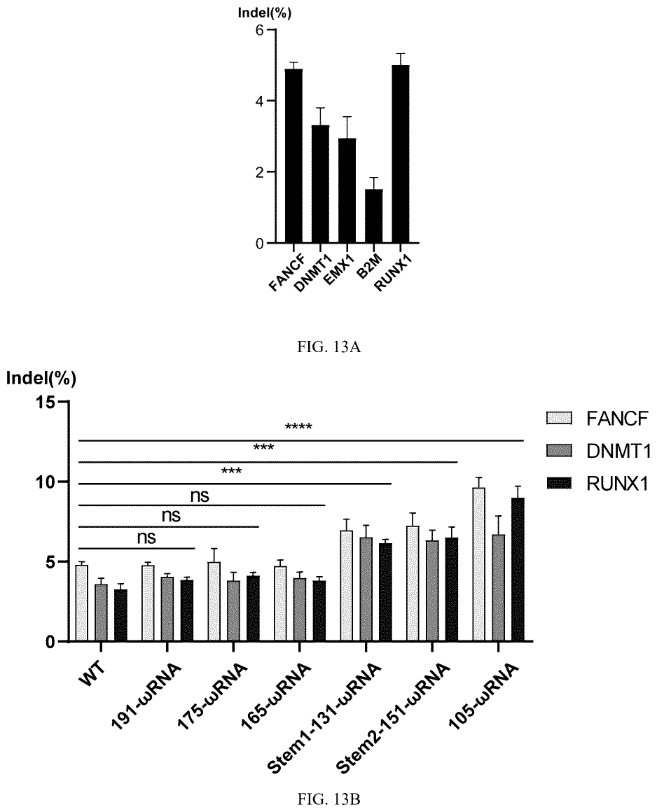

The results are shown in FIG. 12. The activity of the 105-ωRNA was significantly higher than that of the wild type, while the activity of the 97-RNA was lower than that of the wild type. Based on these results, 105-ωRNA is the optimal engineered ωRNA.

### Example 10: Evaluation of the Gene Editing Activity of ISBce3_TnpB Nuclease in HEK293T Cells

In this example, the ISBce3_TnpB sequence was first optimized for eukaryotic cell codons, and SV40 nuclear localization signal (NLS) and NLS nuclear localization signals were added to the N-terminus and C-terminus, respectively. The nucleotide sequence is shown in SEQ ID NO: 36. The nucleotide sequence was cloned into the eukaryotic expression vector and co-transfected with the ωRNA expression vector into HEK 293T cells via liposomes. After 72 h, cells were collected to extract genomic DNA, amplified by PCR, and gene editing activity was detected by high-throughput sequencing.

In this embodiment, the target gene was the human FANCF gene, the TAM sequence was TTAC, the nucleotide sequence of the amplicon is shown in SEQ ID NO: 37, and the sequence of the WT-ωRNA is shown in SEQ ID NO: 38. The DNMT1 gene, with TAM of TTAC, has the nucleotide sequence of the amplicon as shown in SEQ ID NO: 39 and the sequence of the WT-ωRNA as shown in SEQ ID NO: 40; the EMX1 gene, with TAM of TTAC, has the nucleotide sequence of the amplicon as shown in SEQ ID NO: 41 and the sequence of the WT-ωRNA as shown in SEQ ID NO: 42; the RUNX1 gene, with TAM of TTAC, has the nucleotide sequence of the amplicon as shown in SEQ ID NO: 43 and the sequence of the WT-ωRNA as shown in SEQ ID NO: 44; and the B2M gene, with TAM of TTAC, has the nucleotide sequence of the amplicon as shown in SEQ ID NO: 45 and the sequence of the WT-ωRNA as shown in SEQ ID NO: 46.

Specifically, HEK293T cells were seeded when the confluence reached 70-80%. The number of cells seeded in each well of the 12-well plate was 8×10⁴ cells/well. Transfection was performed after 8 h, with each well replaced with Dulbecco's modified eagle medium (DMEM) medium containing 2% fetal bovine serum (FBS) before transfection. The ISBce3_TnpB eukaryotic expression plasmid of 500 ng and the ωRNA expression plasmid of 300 ng were added sequentially to Jetprime Buffer of 200 μL. After mixing well by pipetting, Jetprime of 1.5 μL were added and mixed well by pipetting. The mixture was incubated at room temperature for 10 min, and then added dropwise to the cell supernatant. After culturing at 37° C. for 72 h, the culture medium was discarded, and the cells were resuspended in phosphate buffered saline (PBS) of 100 μL to extract the genomic DNA of the cells. After PCR amplification, the high-throughput sequencing was performed.

The results are shown in FIG. 13A. Gene editing activity was detected at all five target sites, indicating that the novel ISBce3_TnpB nuclease can perform targeted editing of the mammalian genome.

Furthermore, the optimized ωRNA variant described in Example 9, which exhibits enhanced trans-cleavage activity in vitro, was tested at three endogenous target sites in HEK293T cells. As shown in FIG. 13B, the optimized 105-ωRNA has the shortest length and significantly improves the editing efficiency of ISBce3_TnpB.

The embodiments described above are merely preferred embodiments of the present invention and are not intended to limit the scope of the present invention. Various modifications and improvements made by one of ordinary skill in the art to the technical solutions of the present invention without departing from the design spirit of the present invention should fall within the protection scope defined by the claims of the present invention.

What is claimed is:

1. A TnpB nuclease, wherein the TnpB nuclease is one of (A1), (A2), and (A3):
(A1) a TnpB nuclease having the amino acid sequence shown in SEQ ID NO: 1;
(A2) a protein having more than 80% identity with the TnpB nuclease and having an identical biological function as the TnpB nuclease, wherein the protein is obtained by substituting, deleting, and/or adding one or more amino acid residues to the amino acid sequence of the TnpB nuclease; and
(A3) a fusion protein obtained by fusing a protein tag to an N-terminus and/or a C-terminus of (A1) or (A2).

2. A coding gene of the TnpB nuclease according to claim 1.

3. The coding gene according to claim 2, wherein when the amino acid sequence of the TnpB nuclease is as shown in SEQ ID NO: 1, the nucleotide sequence of the coding gene is as shown in SEQ ID NO: 2.

4. A biomaterial, wherein the biomaterial is one of (B1)-(B3):
(B1) a gene expression cassette comprising the coding gene according to claim 2;
(B2) a recombinant vector comprising the gene expression cassette; and
(B3) a recombinant host cell comprising the recombinant vector.

5. A method for preparing the TnpB nuclease according to claim 1, comprising using a coding gene of the TnpB nuclease or a biomaterial, wherein the biomaterial is one of (B1)-(B3):
(B1) a gene expression cassette comprising the coding gene;
(B2) a recombinant vector comprising the gene expression cassette; and
(B3) a recombinant host cell comprising the recombinant vector.

6. A composition for gene editing, comprising the TnpB nuclease according to claim 1 and ωRNA; wherein
the ωRNA comprises a backbone portion and a targeting sequence portion; and
the targeting sequence portion is complementary to a target sequence of a target gene.

7. The composition according to claim 6, wherein the nucleotide sequence of the backbone portion of the ωRNA is as shown in SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35.

8. A gene editing system, comprising a recombinant vector expressing the TnpB nuclease according to claim 1 and a recombinant vector expressing ωRNA; wherein
the ωRNA comprises a backbone portion and a targeting sequence portion;
the targeting sequence portion is complementary to a target sequence of a target gene; and
the nucleotide sequence of the backbone portion of the ωRNA is as shown in SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35.

9. A method for nucleic acid recognition or gene editing for non-disease diagnosis and treatment purpose, comprising using the TnpB nuclease according to claim 1, a composition, or a gene editing system, wherein
the composition for gene editing comprises the TnpB nuclease and ωRNA; wherein
the ωRNA comprises a backbone portion and a targeting sequence portion; and
the targeting sequence portion is complementary to a target sequence of a target gene; and
the gene editing system comprises a recombinant vector expressing the TnpB nuclease and a recombinant vector expressing the ωRNA; and
the nucleotide sequence of the backbone portion of the ωRNA is as shown in SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35.

10. A method of gene editing, comprising a step of transfecting the gene editing system according to claim 8 into a host cell to perform the gene editing.

11. The biomaterial according to claim 4, wherein in the coding gene, when the amino acid sequence of the TnpB nuclease is as shown in SEQ ID NO: 1, the nucleotide sequence of the coding gene is as shown in SEQ ID NO: 2.

12. The method according to claim 5, wherein in the coding gene, when the amino acid sequence of the TnpB nuclease is as shown in SEQ ID NO: 1, the nucleotide sequence of the coding gene is as shown in SEQ ID NO: 2.


SEQUENCE LISTING (48 sequences)

SEQ ID NO: 1 - protein, 369 aa, synthetic construct
MLNNKKQGGENMSQTITVKIKLLPTKEQAVILTEMSRTYISTINNLVSEMVKEKKSTKKTSKNIDVSLPSAVKNQAIKDAKSVFKKAKKRKFETVPVLNKPMCIWNNQNYSFDFTHISLPIMIDGKAKKTPIRALLVDKDNRNFDLLKHKLGTLRITKKANKWIAQISVTIPTTQKTGLKVMGVDLGLKVPAVAVTDDGKVRFFGNGRQNKFKKRKFRSVRKKLAEKKKLNAIRKINNKEQRWMEDKDHKVSRAIVNFAIENKISVIRLEQLANIRQTTRTSRKNEKNLHTWSFYRLSQFIEYKAKLEGIKVEYVNPAYTSQTCPKCSERNKAQDRKYKCKCGFEGHRDIIGAMNIRYAPVVDGKSQSA

SEQ ID NO: 2 - DNA, 1110 bp, synthetic construct
  source 1..1110: mol_type = other DNA
atgttgaacaacaaaaaacaagggggtgaaaatatgtcgcaaacaatcactgtaaaaatcaaattgctacccactaaggaacaggctgtaattttgactgaaatgagtagaacatacatttcaacaataaataatcttgtatctgaaatggtgaaagaaaagaaaagtactaagaaaacaagcaagaacattgatgtatctttgccaagtgcggtaaaaaaccaagcgattaaagatgctaaaagtgttttcaagaaggcaaagaaaagaaaatttgaaaccgttccagtcttaaataaacctatgtgtatttggaacaatcaaaactattcttttgatttcactcacatttccttgccgattatgattgacggtaaagcgaagaaaacacctattcgtgccttattggttgataaagataaccgaaatttcgatttgcttaaacacaagttaggtacattgcgtatcacgaaaaaggcgaataaatggattgcacaaatttccgtaactattcctactactcaaaaaacaggattgaaggtcatgggagtagatttaggactaaaagtacctgctgttgctgtgaccgatgatgggaaagtgcgtttctttggtaatggtagacaaaataaattcaaaaaaaggaaatttcgttcggttcgtaaaaaattagcagaaaagaaaaagttaaatgctatccgtaaaataaacaacaaggaacaacgttggatggaagataaagaccacaaagtaagtcgtgcgattgttaattttgctatagaaaataaaatttctgtcattcgtttagaacaattagcgaatattagacagacgacaagaacaagtcgtaaaaacgaaaagaatttgcacacatggtcattctatcgcctatctcaattcattgaatacaaggctaaattagaaggtattaaagttgaatatgttaatcctgcatacacaagtcaaacatgccccaagtgttccgaaagaaacaaagcacaagacagaaaatacaagtgtaaatgcggattcgaaggacatagagatattattggtgcaatgaatattcgttatgcacctgtggttgatggtaaaagtcaatcagcatag

SEQ ID NO: 3 - DNA, 1107 bp, synthetic construct
  source 1..1107: mol_type = other DNA
atgctgaataacaagaagcagggcggcgaaaatatgagccagaccattaccgttaaaattaagctgctgccgaccaaagaacaggcagttattctgaccgaaatgagccgcacctatattagtaccattaataatctggtgagcgaaatggtgaaagaaaagaaaagtaccaaaaagaccagtaaaaacatcgatgtgagtctgccgagcgcagttaaaaatcaggccattaaggatgccaaaagtgtgtttaaaaaggcaaaaaagcgcaaatttgagaccgtgccggttctgaataagccgatgtgtatttggaataatcagaattatagcttcgactttacccatattagtctgccgattatgattgatggtaaagcaaaaaagaccccgattcgtgccctgctggtggataaagataatcgtaattttgatctgctgaagcataaactgggtaccctgcgtattaccaaaaaagcaaataagtggattgcacagattagtgttaccattccgaccacccagaaaaccggtctgaaagttatgggtgttgatctgggtctgaaagtgccggcagtggccgttaccgatgatggcaaagtgcgctttttcggcaatggtcgccagaataagtttaaaaaacgcaaatttcgcagcgttcgcaaaaaactggcagaaaagaaaaaactgaacgcaattcgcaaaattaacaataaggaacagcgctggatggaagataaagatcataaagtgagccgtgcaattgtgaattttgccattgaaaataagatcagcgtgattcgtctggaacagctggccaatattcgccagaccacccgtaccagccgtaaaaatgaaaagaatctgcatacctggagtttttatcgcctgagtcagtttattgaatataaagcaaagctggaaggtattaaggtggaatatgttaatccggcatataccagtcagacctgtccgaaatgcagtgaacgtaataaggcacaggatcgcaaatataaatgtaaatgtggtttcgaaggccatcgcgatattattggtgcaatgaatattcgctatgcaccggtggtggatggtaaaagccagagtgcc

SEQ ID NO: 4 - DNA, 752 bp, synthetic construct
  source 1..752: mol_type = other DNA
ctgtaacgcagcacagctgaaccgttctgaagaagaagaaagttaatagcagatgccgataccacaagatcagccgtagtgatagaccccacgtaatccgtgtcccaactaatataaaattctcttgctctggatacgttaatatgaccactgggttggtattcctcccgtggcttcaaagcaaaggtaatcatcatcgcacccggatcatcgggggttttaatcgcattgcctccgtagtggaagggtatgtaagagctgcagaactttgatggaaatttatcgataagattgataccatgagcagttacggaaatgtttttaataataggtaatgtgatcggatacgtaacggggctaatatcagatatagatgaacatgcgtctggaagagctgtatctctatcctgaaagcttatctctgcgtggtgagtgggctgcataatggcgttaacaacatgtccgaacttgtgccaatctcggtgttgatgaggattttgatcggagatgttccaggtaggttttaatcctataaacatatattcaatgggccatttaagagcagacattagtttttcatcgtggtggttattgttggtgtgggtcacctgcgttttatggacacgtatcagcgaaaagcgaacgcgttttacaaaaaggttgtgtatttcaggggttacaaacaggttattgatgtaaagttcattattcgtgagcgagatttcattaatgactcctgggataaaccatgg

SEQ ID NO: 5 - RNA, 224 nt, synthetic construct
  source 1..224: mol_type = other RNA
gaatattcgttatgcacctgtggttgatggtaaaagtcaatcagcatagggaactatatgttctgccctatgaggggtaatgagataccctaatcttgaggactgttcaaaacagaaatggactgcgaacgcttagtcactcaagaatcccacccgttaaaccgtaaggtttagggcttgcgtctttagacgtgggagtctcaactttgctttgaagccacggg

SEQ ID NO: 6 - DNA, 20 bp, synthetic construct
  source 1..20: mol_type = other DNA
ctgtaacgcagcacagctga

SEQ ID NO: 7 - DNA, 20 bp, synthetic construct
  source 1..20: mol_type = other DNA
ccatggtttatcccaggagt

SEQ ID NO: 8 - DNA, 20 bp, synthetic construct
  source 1..20: mol_type = other DNA
cccgtggcttcaaagcaaag

SEQ ID NO: 9 - DNA, 20 bp, synthetic construct
  source 1..20: mol_type = other DNA
cccgtggcttcaaagcaaag

SEQ ID NO: 10 - DNA, 20 bp, synthetic construct
  source 1..20: mol_type = other DNA
gaataccaacccagtggtca

SEQ ID NO: 11 - DNA, 59 bp, synthetic construct
  source 1..59: mol_type = other DNA
tgaccactgggttggtattcctcccgtggcttcaaagcaaaggtattcatcatcgcacc

SEQ ID NO: 12 - DNA, 59 bp, synthetic construct
  source 1..59: mol_type = other DNA
ttccactacggaggcaatgcgattaaaacccccgatgatccgggtgcgatgatgaatac

SEQ ID NO: 13 - DNA, 20 bp, synthetic construct
  source 1..20: mol_type = other DNA
ttgcctccgtagtggaaggg

SEQ ID NO: 14 - DNA, 21 bp, synthetic construct
  source 1..21: mol_type = other DNA
ccatggtttatcccaggagtc

SEQ ID NO: 15 - RNA, 204 nt, synthetic construct
  source 1..204: mol_type = other RNA
gaatattcgttatgcacctgtggttgatggtaaaagtcaatcagcatagggaactatatgttctgccctatgaggggtaatgagataccctaatcttgaggactgttcaaaacagaaatggactgcgaacgcttagtcactcaagaatcccacccgttaaaccgtaaggtttagggcttgcgtctttagacgtgggagtctcaa

SEQ ID NO: 16 - RNA, 191 nt, synthetic construct
  source 1..191: mol_type = other RNA
gcacctgtggttgatggtaaaagtcaatcagcatagggaactatatgttctgccctatgaggggtaatgagataccctaatcttgaggactgttcaaaacagaaatggactgcgaacgcttagtcactcaagaatcccacccgttaaaccgtaaggtttagggcttgcgtctttagacgtgggagtctcaa

SEQ ID NO: 17 - RNA, 175 nt, synthetic construct
  source 1..175: mol_type = other RNA
gtaaaagtcaatcagcatagggaactatatgttctgccctatgaggggtaatgagataccctaatcttgaggactgttcaaaacagaaatggactgcgaacgcttagtcactcaagaatcccacccgttaaaccgtaaggtttagggcttgcgtctttagacgtgggagtctcaa

SEQ ID NO: 18 - RNA, 165 nt, synthetic construct
  source 1..165: mol_type = other RNA
atcagcatagggaactatatgttctgccctatgaggggtaatgagataccctaatcttgaggactgttcaaaacagaaatggactgcgaacgcttagtcactcaagaatcccacccgttaaaccgtaaggtttagggcttgcgtctttagacgtgggagtctcaa

SEQ ID NO: 19 - RNA, 156 nt, synthetic construct
  source 1..156: mol_type = other RNA
gggaactatatgttctgccctatgaggggtaatgagataccctaatcttgaggactgttcaaaacagaaatggactgcgaacgcttagtcactcaagaatcccacccgttaaaccgtaaggtttagggcttgcgtctttagacgtgggagtctcaa

SEQ ID NO: 20 - RNA, 140 nt, synthetic construct
  source 1..140: mol_type = other RNA
gccctatgaggggtaatgagataccctaatcttgaggactgttcaaaacagaaatggactgcgaacgcttagtcactcaagaatcccacccgttaaaccgtaaggtttagggcttgcgtctttagacgtgggagtctcaa

SEQ ID NO: 21 - RNA, 133 nt, synthetic construct
  source 1..133: mol_type = other RNA
gaggggtaatgagataccctaatcttgaggactgttcaaaacagaaatggactgcgaacgcttagtcactcaagaatcccacccgttaaaccgtaaggtttagggcttgcgtctttagacgtgggagtctcaa

SEQ ID NO: 22 - RNA, 121 nt, synthetic construct
  source 1..121: mol_type = other RNA
gataccctaatcttgaggactgttcaaaacagaaatggactgcgaacgcttagtcactcaagaatcccacccgttaaaccgtaaggtttagggcttgcgtctttagacgtgggagtctcaa

SEQ ID NO: 23 - RNA, 110 nt, synthetic construct
  source 1..110: mol_type = other RNA
cttgaggactgttcaaaacagaaatggactgcgaacgcttagtcactcaagaatcccacccgttaaaccgtaaggtttagggcttgcgtctttagacgtgggagtctcaa

SEQ ID NO: 24 - DNA, 500 bp, synthetic construct
  source 1..500: mol_type = other DNA
atccacaccatctgcattgtcttactagcccccctgtgtagaccactggttcacctttgacgcatgtccagttgccccccaacttacagtagaataaatcctcattttccaccgtggtggtcacgcaatccgttctatgcgaaggcttatctctcctaaaggtctttaccacttccgttctcagagttgtagggctcaccgctgtgcactctacgacacccgtccaccctattgggcagacaagatagaaggcactgccgttcaataaggttgtattgtacttccccttgacaacaggactagtatcgaaagggcacagcccgaacccaaagtcgtctcccatttcctcagtagatgggttggtcccgtcgaacaagagctcaaatgttacagacgtgggtagagccctcttgtgtaatgatgccaagtacctcctgctgaccacgctgagtgcagtgactttaaaggagcccgccacgcaaatagccttgacggtcccg

SEQ ID NO: 25 - DNA, 20 bp, synthetic construct
  source 1..20: mol_type = other DNA
caactctgagaacggaagtg

SEQ ID NO: 26 - RNA, 150 nt, synthetic construct
  source 1..150: mol_type = other RNA
atcagcatagggaactatatgttctgccctatgaggggtaatgagataccctaatcttgaggactgttgaaataacgcttagtcactcaagaatcccacccgttaaaccgtaaggtttagggcttgcgtctttagacgtgggagtctcaa

SEQ ID NO: 27 - RNA, 139 nt, synthetic construct
  source 1..139: mol_type = other RNA
atcagcatagggaactatatgttctgccctatgaggggtaatgagataccctaatcttgaggactgaaaagtcactcaagaatcccacccgttaaaccgtaaggtttagggcttgcgtctttagacgtgggagtctcaa

SEQ ID NO: 28 - RNA, 138 nt, synthetic construct
  source 1..138: mol_type = other RNA
atcagcatagggaactatatgttctgccctatgaggggtaatgagataccctaatcttgaggacgaaatgtcactcaagaatcccacccgttaaaccgtaaggtttagggcttgcgtctttagacgtgggagtctcaa

SEQ ID NO: 29 - RNA, 131 nt, synthetic construct
  source 1..131: mol_type = other RNA
atcagcatagggaactatatgttctgccctatgaggggtaatgagataccctaatcttgaggatcactcaagaatcccacccgttaaaccgtaaggtttagggcttgcgtctttagacgtgggagtctcaa

SEQ ID NO: 30 - RNA, 155 nt, synthetic construct
  source 1..155: mol_type = other RNA
atcagcatagggaactatatgttctgccctatgaggggtaatgagataccctaatcttgaggactgttcaaaacagaaatggactgcgaacgcttagtcactcaagaatcccacccccgtaagggggcttgcgtctttagacgtgggagtctcaa

SEQ ID NO: 31 - RNA, 151 nt, synthetic construct
  source 1..151: mol_type = other RNA
atcagcatagggaactatatgttctgccctatgaggggtaatgagataccctaatcttgaggactgttcaaaacagaaatggactgcgaacgcttagtcactcaagaatcccacccgtaagggcttgcgtctttagacgtgggagtctcaa

SEQ ID NO: 32 - RNA, 149 nt, synthetic construct
  source 1..149: mol_type = other RNA
atcagcatagggaactatatgttctgccctatgaggggtaatgagataccctaatcttgaggactgttcaaaacagaaatggactgcgaacgcttagtcactcaagaatcccacccgttaaaccgtaaggtttagggtgggagtctcaa

SEQ ID NO: 33 - RNA, 131 nt, synthetic construct
  source 1..131: mol_type = other RNA
atcagcatagggaactatatgttctgccctatgaggggtaatgagataccctaatcttgaggactgttcaaaacagaaatggactgcgaacgcttagtcactcaagaatcccacgtaagtgggagtctcaa

SEQ ID NO: 34 - RNA, 105 nt, synthetic construct
  source 1..105: mol_type = other RNA
atcagcatagggaactatatgttctgccctatgaggggtaatgagataccctaatcttgaggatcactcaagaatcccacccgtaatttagggtgggagtctcaa

SEQ ID NO: 35 - RNA, 97 nt, synthetic construct
  source 1..97: mol_type = other RNA
atcagcatagggaactatatgttctgccctatgaggggtaatgagataccctaatcttgaggatcactcaagaatcccacgtaagtgggagtctcaa

SEQ ID NO: 36 - DNA, 1179 bp, synthetic construct
  source 1..1179: mol_type = other DNA
atggccccaaagaagaagcggaaggtcctgaacaacaagaagcagggcggagaaaacatgtctcagacaatcacagtgaagatcaagctgctgcctacaaaggaacaagccgtgatcctgacagagatgagcagaacctacatcagcaccatcaacaacctggtgtccgagatggtgaaagaaaagaagagcaccaagaaaaccagcaagaatatcgacgtgtctctgcctagcgccgtgaagaatcaggctatcaaggacgccaagagcgttttcaagaaagcaaagaagcggaagtttgagacagtgccagtgctgaacaagcctatgtgcatctggaacaatcaaaactacagctttgacttcacccacatcagcctgcccatcatgatcgatggcaaggccaagaaaacccctatcagagccctgctcgtggacaaggataacagaaatttcgacctgctgaagcacaagctcggcaccctgagaatcaccaagaaggccaacaagtggatcgcccagatcagcgtgaccattcctaccacccagaaaacaggcctgaaagtgatgggcgtggacctgggcctgaaggtccccgctgtggccgtgacagacgacggcaaggtgcggttcttcggcaacggcagacagaacaaattcaagaaaagaaagttccggagcgtcagaaagaagctggccgagaagaaaaagctgaatgccattcggaagatcaacaacaaagaacagcggtggatggaagataaggaccataaggtgtccagagccatcgtgaacttcgccattgagaacaagatctccgtcatccggctggaacagctggctaatatcaggcagaccaccagaacaagccgcaagaacgagaaaaacctgcacacctggtccttctacagactgagccaatttatcgagtacaaggccaaactggagggcatcaaggtggaatatgtgaaccccgcctacacctctcagacctgtcctaagtgcagcgagcggaacaaggctcaggacagaaagtacaagtgtaaatgcggcttcgagggccacagagatatcatcggagccatgaacatcagatacgcccctgtggtggatggaaagtctcagagcgccaagcgtcctgctgctactaagaaagctggtcaagctaagaaaaagaaa

SEQ ID NO: 37 - DNA, 132 bp, synthetic construct
  source 1..132: mol_type = other DNA
actgtgacgtcctgctctctctgcgcctgctggagaaccgggccctcggggatgcagctcgttaccacctggtgcagcaactctttcccggcccgggcgtccgggacgccgatgaggagacactccaagaga

SEQ ID NO: 38 - RNA, 224 nt, synthetic construct
  source 1..224: mol_type = other RNA
gaatattcgttatgcacctgtggttgatggtaaaagtcaatcagcatagggaactatatgttctgccctatgaggggtaatgagataccctaatcttgaggactgttcaaaacagaaatggactgcgaacgcttagtcactcaagaatcccacccgttaaaccgtaaggtttagggcttgcgtctttagacgtgggagtctcaacacctggtgcagcaactctt

SEQ ID NO: 39 - RNA, 188 nt, synthetic construct
  source 1..188: mol_type = other RNA
agtctccgtgaacgttcccttagcactctgccacttattgggtcagctgttaacatcagtacgttaatgtttcctgatggtccatgtctgttactcgcctgtcaagtggcgtgacaccgggcgtgttccccagagtgacttttccttttatttcccttcagctaaaataaaggaggaggaagctgcta

SEQ ID NO: 40 - RNA, 224 nt, synthetic construct
  source 1..224: mol_type = other RNA
gaatattcgttatgcacctgtggttgatggtaaaagtcaatcagcatagggaactatatgttctgccctatgaggggtaatgagataccctaatcttgaggactgttcaaaacagaaatggactgcgaacgcttagtcactcaagaatcccacccgttaaaccgtaaggtttagggcttgcgtctttagacgtgggagtctcaatcgcctgtcaagtggcgtga

SEQ ID NO: 41 - RNA, 175 nt, synthetic construct
  source 1..175: mol_type = other RNA
tggggtggtagattccggctctgagggccagtgggggctggtagagcaaacgcgttcagggcctgggagcctggggtggggtactggtggagggggtcaagggtaattcattaactcctctcttttgttgggggaccctggtctctacctccagctccacagcaggagaaacagg

SEQ ID NO: 42 - RNA, 224 nt, synthetic construct
  source 1..224: mol_type = other RNA
gaatattcgttatgcacctgtggttgatggtaaaagtcaatcagcatagggaactatatgttctgccctatgaggggtaatgagataccctaatcttgaggactgttcaaaacagaaatggactgcgaacgcttagtcactcaagaatcccacccgttaaaccgtaaggtttagggcttgcgtctttagacgtgggagtctcaaccttgaccccctccaccagt

SEQ ID NO: 43 - RNA, 166 nt, synthetic construct
  source 1..166: mol_type = other RNA
tcccagaggtatccagcagaggggagaagaaagagagatgtagggctagaggggtgaggctgaaacagtgacctgtcttggttttcgctccgaaggtaaaagaaatcattgagtcccccgccttcagaagagggtgcattttcaggaggaagcgatggcttcagac

SEQ ID NO: 44 - RNA, 224 nt, synthetic construct
  source 1..224: mol_type = other RNA
gaatattcgttatgcacctgtggttgatggtaaaagtcaatcagcatagggaactatatgttctgccctatgaggggtaatgagataccctaatcttgaggactgttcaaaacagaaatggactgcgaacgcttagtcactcaagaatcccacccgttaaaccgtaaggtttagggcttgcgtctttagacgtgggagtctcaacttcggagcgaaaaccaaga

SEQ ID NO: 45 - RNA, 134 nt, synthetic construct
  source 1..134: mol_type = other RNA
cactaggaccttctctgagctgtcctcaggatgcttttgggactatttttcttacccagagaatggagaaaccctgcagggaattcccaagctgtagttataaacagaagttctccttctgctaggtagcattc

SEQ ID NO: 46 - RNA, 224 nt, synthetic construct
  source 1..224: mol_type = other RNA
gaatattcgttatgcacctgtggttgatggtaaaagtcaatcagcatagggaactatatgttctgccctatgaggggtaatgagataccctaatcttgaggactgttcaaaacagaaatggactgcgaacgcttagtcactcaagaatcccacccgttaaaccgtaaggtttagggcttgcgtctttagacgtgggagtctcaaccagagaatggagaaaccct

SEQ ID NO: 47 - DNA, 12 bp, synthetic construct
  source 1..12: mol_type = other DNA
gtatccagtgcg

SEQ ID NO: 48 - RNA, 12 nt, synthetic construct
  source 1..12: mol_type = other RNA
gtatccagtgcg